(19) United States
(12) Patent Application Publication
(10) Pub. No.: US 2026/0151043 A1
(43) Pub. Date: Jun. 4, 2026

(54) APPARATUS FOR IMAGE-BASED CARDIOVASCULAR BIOMETRIC INFORMATION ESTIMATION OR METHOD THEREFOR

(71) Applicant: LG ELECTRONICS INC., Seoul (KR)

(72) Inventors: Byungki CHAE, Seoul (KR); Byunggoo KONG, Seoul (KR); Hyounggil YOON, Seoul (KR); Taehyung LEE, Seoul (KR); Hangjin BYEON, Seoul (KR)

(73) Assignee: LG ELECTRONICS INC., Seoul (KR)

(21) Appl. No.: 19/221,978

(22) Filed: May 29, 2025

(30) Foreign Application Priority Data
Dec. 3, 2024 (WO) PCT/KR2024/019543

(51) Int. Cl. A61B 5/026; A61B 5/0295; G06V 10/30; G06V 10/56; G06V 10/75; G06V 40/10; G06V 40/16; G16H 50/30

(52) U.S. Cl. CPC A61B 5/0261; A61B 5/0295; G06V 10/30; G06V 10/56; G06V 10/75; G06V 40/15; G06V 40/166; G06V 40/171; G16H 50/30; A61B 2576/00

(57) ABSTRACT

Proposed is an apparatus for performing image-based cardiovascular biometric information estimation. The apparatus includes a memory that stores data for the cardiovascular biometric information estimation, and a processor that estimates cardiovascular biometric information of an object in an image obtained using the data for the cardiovascular biometric information estimation.

This application claims the benefit of International Patent Application No. PCT/KR2024/019543, filed on Dec. 3, 2024, which is hereby incorporated by reference as if fully set forth herein.

### BACKGROUND

### Field

The present disclosure relates to an apparatus for image-based cardiovascular biometric information estimation or a method therefor, and more particularly, to an apparatus equipped to estimate cardiovascular biometric information of an object identified from an image and additionally output the estimated cardiovascular biometric information or a method therefor.

### Discussion of the Related Art

Remote photoplethysmography (rPPG) refers to a technology for measuring a physiological signal such as a heart rate in a non-contact manner. This technology is based on a principle of tracking a blood flow by analyzing a change in light reflected from skin.

A specific color change (mainly a green channel) in an image captured via a camera through a skin surface is analyzed, and the color change occurs as an amount of skin blood changes based on a heartbeat.

From the captured image data, noise (a movement, a lighting change, and the like) is removed using a sophisticated algorithm and periodic signals based on the blood flow are extracted.

The rPPG has high user convenience because it does not need to attach a sensor to the skin, and enables various applications as measurement is available even with a smartphone, a webcam, a CCTV, and the like. In addition, the rPPG is safe and simple because a body is not directly touched during the measurement of the bio-signal.

Accordingly, recently, the rPPG is used or applied in a healthcare field for monitoring health by measuring the heart rate, a blood pressure, a stress level, and the like, a fitness field for analyzing the heart rate, a physical condition, and the like during exercise, a security monitoring and emotional state analysis field for checking stress or tension, and the like.

The rPPG technology is gradually developing, and is evolving into a more accurate, real-time applicable system combined with artificial intelligence.

### SUMMARY

The present disclosure is to propose a method for reducing performance deterioration in a high heart rate section and a low heart rate section in obtaining a cardiovascular bio-signal such as a heart rate using rPPG.

In addition, the present disclosure is to propose a method for reducing performance deterioration resulted from image pixel or region size limitations in obtaining a cardiovascular bio-signal such as a heart rate using rPPG for multiple objects.

The problems to be solved by the present disclosure are not limited to the problems described above, and other problems not mentioned will be clearly understood by those skilled in the art to which the present disclosure pertains from the following description.

According to an embodiment of the present disclosure, an apparatus for performing image-based cardiovascular biometric information estimation is proposed. The apparatus includes a memory that stores data for estimating the cardiovascular biometric information, and a processor that estimates cardiovascular biometric information of an object in an obtained image using the data for estimating the cardiovascular biometric information.

In one implementation, the processor may estimate cardiovascular biometric information of a plurality of objects in the obtained image.

In one implementation, the processor may output the estimated cardiovascular biometric information of the object via a human-machine interface.

In one implementation, the processor may crop an image region corresponding to each of the plurality of objects, and/or scale up the cropped image region.

In one implementation, the processor may identify the plurality of objects and output the estimated cardiovascular biometric information of the identified objects via a human-machine interface.

In one implementation, the processor may search for information matching the plurality of objects from user information stored in the memory.

In one implementation, the processor may transmit or output a notification message in response to that cardiovascular biometric information of one of the plurality of objects exceeds a preset threshold value or deviates from a threshold range.

In one implementation, the data for estimating the cardiovascular biometric information may include data obtained by learning RGB data of an image and photoplethysmography (PPG) data corresponding to the RGB data. The RGB data of the image may include data obtained by preprocessing the RGB data of the image. Further, the PPG data may correspond to a particular value of the cardiovascular biometric information.

In one implementation, the data for estimating the cardiovascular biometric information estimation may include data obtained by learning virtual RGB data for a preset value or a preset range of cardiovascular biometric information and virtual remote photoplethysmography (rPPG) data corresponding to the virtual RGB data.

According to another embodiment of the present disclosure, a method for performing image-based cardiovascular biometric information estimation is proposed. The method includes obtaining an image, detecting an object from the obtained image and obtaining a signal for a face region of the detected object, and estimating cardiovascular biometric information of the detected object based on the obtained signal.

According to still another embodiment of the present disclosure, a computer-readable medium storing code to execute, by a computer or a processor, the method for performing the image-based cardiovascular biometric information estimation is proposed.

The technical solutions above are only some of embodiments of the present disclosure, and various embodiments reflecting the technical features of the present disclosure may be derived and understood by those skilled in the art based on the detailed description of the present disclosure to be described below.

The present disclosure has the following technical effects.

The present disclosure may reduce the performance deterioration in the high heart rate section and the low heart rate section in obtaining the cardiovascular bio-signal such as the heart rate using the rPPG.

In addition, the present disclosure may reduce the performance deterioration resulted from the image pixel or region size limitations in obtaining the cardiovascular bio-signal such as the heart rate using the rPPG for the multiple objects.

The effects according to the present disclosure are not limited to the above-mentioned effects, and other effects not mentioned may be clearly understood by those skilled in the art to which the present disclosure pertains from the following detailed description of the present disclosure.

### BRIEF DESCRIPTION OF THE DRAWINGS

The accompanying drawings, which are included as part of the detailed description to help understand the present disclosure, provide the embodiments of the present disclosure and illustrate the technical concepts of the present disclosure together with the detailed description.

FIG. 1 is a diagram for illustrating a heart rate detection principle using PPG.

FIG. 2 is a diagram for illustrating a principle of rPPG.

FIG. 3 shows heart rate distributions during a normal rest period of women and men.

FIG. 4 shows a result of heart rate estimation according to prior art.

FIG. 5 shows a process of augmenting an rPPG signal in a preset heart rate range according to the present disclosure.

FIG. 6 is a flowchart of image-based heart rate estimation according to the present disclosure.

FIG. 7 illustrates a learning process of a model for image-based heart rate estimation according to the present disclosure.

FIG. 8 shows a result of heart rate estimation according to the present disclosure.

FIG. 9 is a flowchart of image-based heart rate estimation for multiple objects according to the present disclosure.

FIG. 10 illustrates a block diagram of an apparatus for image-based cardiovascular-related biometric information estimation according to the present disclosure.

### DESCRIPTION OF SPECIFIC EMBODIMENTS

Hereinafter, embodiments of the present disclosure will be described in detail with reference to the attached drawings. In this specification, the same or equivalent components will be provided with the same reference numbers, and description thereof will not be repeated. The suffixes “module” and “unit” used for the components in the following description are assigned or used for convenience of description, and do not inherently have distinct meanings or roles. The suffixes are employed solely for ease of reference and should not be considered to convey unique distinctions in meaning or function. If it is deemed that detailed descriptions of the related art obscure the gist of the embodiments disclosed in this specification, the detailed descriptions will be omitted. It should be understood that the attached drawings are merely to provide better understanding of the embodiments disclosed herein and the technical concepts of the present disclosure are not limited to the attached drawings. Thus, the present disclosure should be construed to encompass all alterations, equivalents, and alternatives within the scope of the concepts and technologies disclosed in the present disclosure.

While terms such as “first,” “second,” and so on may be used to describe various components, but the aforementioned components are not limited by these terms. The above terms are used only to distinguish one component from another.

When a component is mentioned to be “connected” or “coupled” to another component, it may be directly connected or coupled to the other component, but it should be understood that there could also be other components in between. On the other hand, when a component is mentioned to be “directly connected” or “directly coupled” to another component, it should be understood that there are no other components in between.

Unless singular expressions clearly indicate otherwise in context, the singular expressions encompass plural expressions.

In the present disclosure, terms such as “comprises” or “includes” are intended to indicate the presence of features, numbers, steps, operations, components, parts, or combinations thereof as specified in the specification, rather than to preclude the presence or possibility of one or more other features, numbers, steps, operations, components, parts, or combinations thereof.

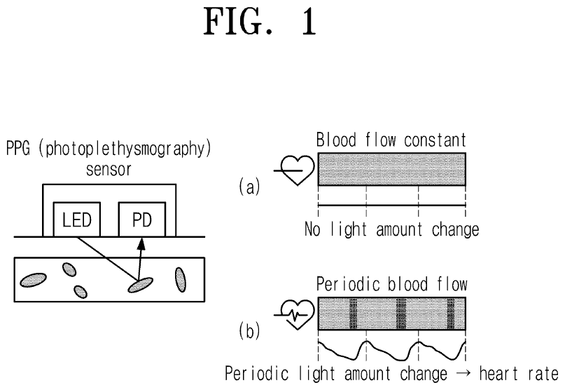

FIG. 1 is a diagram for illustrating a heart rate detection principle using photoplethysmography (PPG).

The PPG is a non-invasive optical technology and is a physiological signal obtaining scheme that measures blood flow dynamics in a body caused by a change in a blood volume. The PPG is mainly based on a principle of irradiating infrared (IR) or visible light to skin and then detecting a change in reflected or transmitted light. Referring to FIG. 1, a process of obtaining an analog signal by detecting light reflected from hemoglobin in blood by a photodetector PD when light is irradiated toward the skin from a light source LED is illustrated.

This uses a principle of detecting periodic changes in the blood flow based on a heartbeat using light absorption characteristics of the blood.

In the present document, only a heart rate will be mentioned as cardiovascular biometric information obtained from the PPG or rPPG, but the present invention may be expanded to estimate following biometric information in addition to the heart rate, and the scope of the present disclosure should include the estimation of the following biometric information.
Heart rate variability (HRV)
Blood oxygen saturation (SpO₂)
Respiratory rate (RR)
Pulse wave velocity (PWV)
Blood pressure (BP)
Vascular compliance & volume changes
Stress index
Cardiac output (CO)
Derived cardiac indices
Pulse wave analysis-based indicator

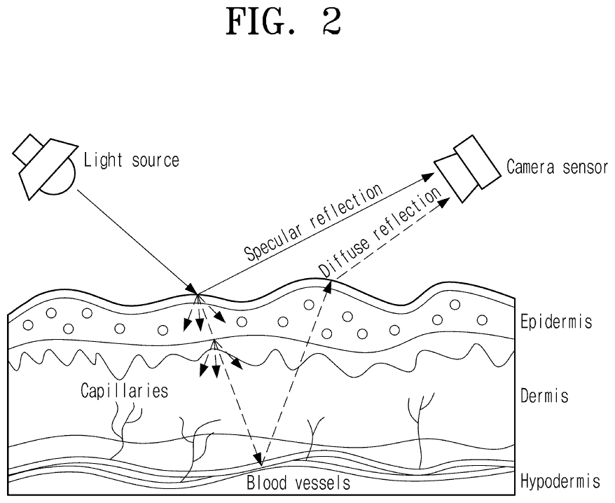

FIG. 2 is a diagram for illustrating a principle of rPPG. Basically, the rPPG is the same as the PPG, but is different in that it is of a non-contact type. Referring to FIG. 2, it is identified that both a light source and a sensor (a camera) are separated from the skin.

In the same manner as in the PPG, in addition to a component in external light reflected from a skin surface, a component absorbed and scattered within the skin and reflected to the camera (diffused reflection) may be monitored to detect heartbeat information based on a minute change in the blood flow on the skin surface.

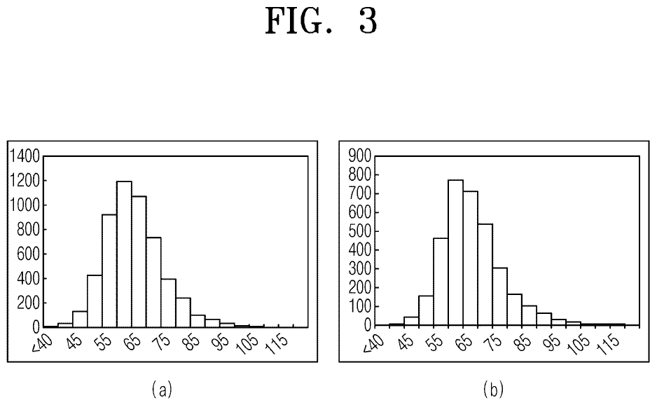

FIG. 3 shows heart rate distributions during a normal rest period of women and men.

(a) in FIG. 3 shows the heart rate distribution during the rest period of the women, and (b) in FIG. 3 shows the heart rate distribution during the rest period of the men.

It may be seen that the women have a lower average heart rate distribution than the men.

However, in a section in which the heart rate distribution is sparse, for example, a section in which a heart rate per minute is equal to or lower than 50 and a section in which the heart rate per minute is equal to or higher than 80, there is a high probability that the heart rate estimation based on the image-based rPPG is inaccurate. This is because, in the above heart rate per minute sections, the rPPG-based estimation, which derives an rPPG signal from RGB data extracted from an image and relies on an inference model through a training process, lacks sufficient training data.

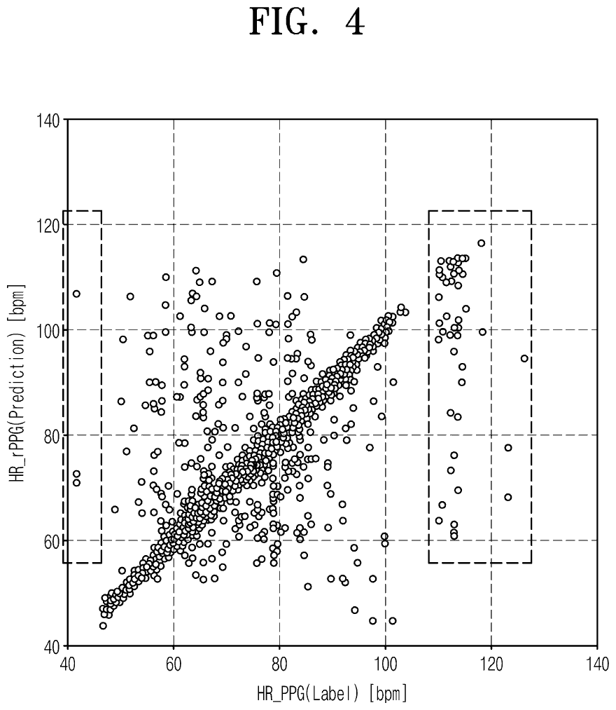

FIG. 4 shows a result of heart rate estimation according to prior art. FIG. 4 shows a result of rPPG-based heart rate estimation of an object obtained from a plurality of images. In FIG. 4, an x-axis represents an input of an PPG signal (a data set) corresponding to a known heart rate, and a y-axis represents an inference result obtained by processing the input in the inference model, that is, an estimated value of the heart rate.

Because the x-axis and the y-axis in FIG. 4 have the same dimension and scale, points distributed close to a straight line with a slope of 1 indicate accurate estimation results, and points distributed far from the straight line indicate errors.

Referring to FIG. 4, error data is identified in a low heart rate section (equal to or lower than about 50 bpm) and a high heart rate section (equal to or higher than about 110 bpm), indicated by a rectangle.

In the present disclosure, it is intended to propose configuration, design, or training of the inference model for obtaining a better estimation result in the above heart rate sections, and heart rate estimation using the same.

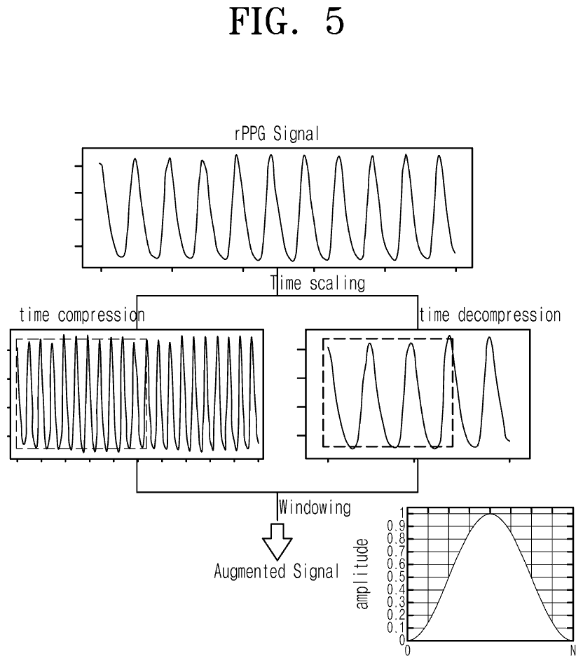

FIG. 5 shows a process of augmenting an rPPG signal in a preset heart rate range according to the present disclosure.

As described above with reference to the estimation result shown in FIG. 4, the heart rate distribution shown in FIG. 3 results in a decrease in an estimation accuracy in the relatively low or high heart rate range. Accordingly, in the present proposal, a method for increasing the estimation accuracy in the relatively low or high heart rate range will be described.

An rPPG signal shown at the top of FIG. 5 is provided. The rPPG signal, as a reference signal, may be an rPPG signal (hereinafter, referred to as a “reference rPPG signal”) corresponding to a preset heart rate (or a preset heart rate range). The reference rPPG signal shown is expressed in a time domain, and thus an x-axis represents time. For example, the preset heart rate may be 60 bpm.

The reference rPPG signal may include data in which data obtained from the image (i.e., data obtained by preprocessing RGB data of at least a partial region of the image) and the PPG signal are paired with each other. For example, when the preset heart rate is 60 bpm, data obtained from the image obtained by measuring the 60 bpm and a PPG signal at that time may be paired with each other.

The preprocessing of the RGB data of the image may include a process of obtaining an alternating current (AC) signal in a time domain extracted via filtering from an RGB signal representing R, G, and B pixel values in the time domain for a region of interest (e.g., a face of the object or the like) in the image.

In addition, signal analysis processing such as feature extraction or noise removal may be performed on the filtered RGB signal via synthesization, calculation, or reconstruction of the signal for each channel (i.e., for each of R, G, and B). Signal analysis processing techniques may include independent component analysis (ICA), principal component analysis (PCA), plane-orthogonal-to-skin (POS), chromatography-based method (CHROM), green channel method (GREEN), peak-to-valley-based method (PVB), spatial subspace rotation (SSR), local group invasion (LGI), and the like, but the present disclosure may not be limited thereto.

When a Fourier transform is performed on the reference rPPG signal in the time domain, heart rate information may be obtained, which corresponds to information of the preset heart rate or the preset heart rate range described above.

The reference rPPG signal is time-scaled in the time domain to obtain a virtual rPPG signal of a heart rate or a heart rate range different from the information of the preset heart rate or the preset heart rate range. More specifically, the reference rPPG signal is compressed or decompressed (or time-stretched) in the time domain to obtain the virtual rPPG signal of the heart rate or the heart rate range different from the information of the preset heart rate or the preset heart rate range.

When the reference rPPG signal is compressed in the time domain, a virtual rPPG signal of a heart rate or a heart rate range higher than the preset heart rate or the preset heart rate range may be obtained. In addition, when the reference rPPG signal is decompressed in the time domain, a virtual rPPG signal of a heart rate or a heart rate range lower than the preset heart rate or the preset heart rate range may be obtained.

The obtained virtual rPPG signal may be processed using a window (or a window function) to obtain an augmented signal or data. The data processing using the window or the window function may improve accuracy of frequency analysis by mitigating a boundary effect. As the window function, a Hann window, a Hamming window, or the like may be used, but the present disclosure may not be limited thereto.

The process of obtaining the augmented signal (or augmented data) from the reference rPPG signal (or reference rPPG data) is represented in a mathematical formula as follows.

$$x ( t ) = PPG / px paired Signal \qquad [ Mathematical Formula 1 ]$$

$${x}_{a} ( t ) = x ( at )$$

$$Sig {( n )}_{Aug} = {x}_{a} ( n + kM ) \cdot w ( n ) , 0 \leq n < N$$

where x(t) denotes a signal in which PPG and px are paired with each other, px denotes a signal obtained by preprocessing an image signal (or data), and

xₐ(t) denotes a signal obtained by time-scaling x(t). When a>1, a time axis is compressed to increase a frequency of the signal (that is, a signal of a high heart rate), and when 0<a<1, the time axis is expanded to decrease the frequency (that is, a signal of a low heart rate).

Further, N denotes a window magnitude, M denotes a stride, and k denotes a window index,

n denotes a time index within a single window, w denotes the window function (Han, Hamming, and the like), and

Sig(n)_{Aug} denotes the augmented signal or the augmented data. The augmented signal or the augmented data may be used in the training process of the model for the image-based heart rate estimation as will be described later, and specifically, may be used as a correct answer of the estimation model.

The reference rPPG and the augmented data composed of PPG, px, and heart rate information may be represented as follows.

TABLE 1

| | | | Reference/augmented |
|---|---|---|---|
| PPG | px | Heart rate (bpm) | data |
| PPG(x1) | px1 | 55 | Reference data |
| PPG(x2) | px2 | 60 | Reference data |
| PPG(x3) | px3 | 65 | Reference data |
| . . . | . . . | . . . | . . . |
| PPG(xn-1) | pxn-1 | 100 | Augmented data |
| PPG(xn) | pxn | 110 | Augmented data |

The augmented signal or the augmented data obtained as such may be used as the training data of the image-based heart rate inference model. That is, the obtained augmented signal or the augmented data is data used for the heart rate estimation.

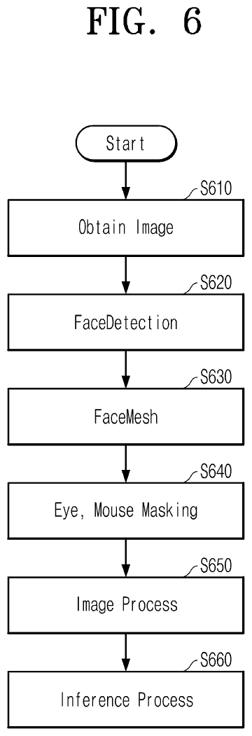

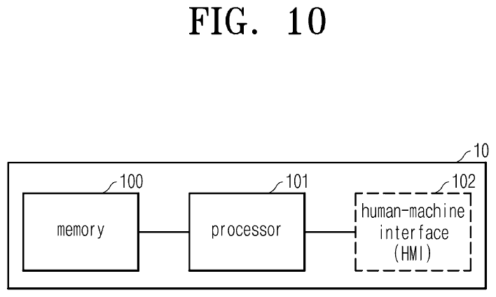

FIG. 6 is a flowchart of image-based heart rate estimation according to the present disclosure. The image-based heart rate estimation according to the present disclosure may be performed by a heart rate estimation apparatus or may be performed by a processor of the heart rate estimation apparatus. A description of the heart rate estimation apparatus will be made later with reference to FIG. 10. Hereinafter, it will be simply described that an apparatus 10 performs the heart rate estimation.

The apparatus 10 may obtain the image (S610). The image may be obtained via an image sensor such as the camera.

The apparatus 10 may detect a face region of the object from the obtained image (S620).

The apparatus 10 may extract specific landmarks (main feature points) from the face by performing a face mesh (S630). The face mesh, as a technology for modeling a geometrical structure of the face by extracting the specific landmarks of the face from computer vision with high precision, may represent the main feature points of the face in a 3D or 2D space.

The apparatus 10 may set the region of interest from the result of the face mesh, i.e., the main feature points of the face, and mask a noise-intensive region (e.g., eyes, mouth, and the like) (S640).

The apparatus 10 may perform image data processing on the masked region of interest (S650). The image data processing may include the pre-processing of the RGB data of the image described above with reference to FIG. 5.

The apparatus 10 may obtain an inference result using the image data processed via the inference model as an input (S660). The inference model may include a model including the augmented signal or the augmented data described above or trained using the augmented signal or the augmented data.

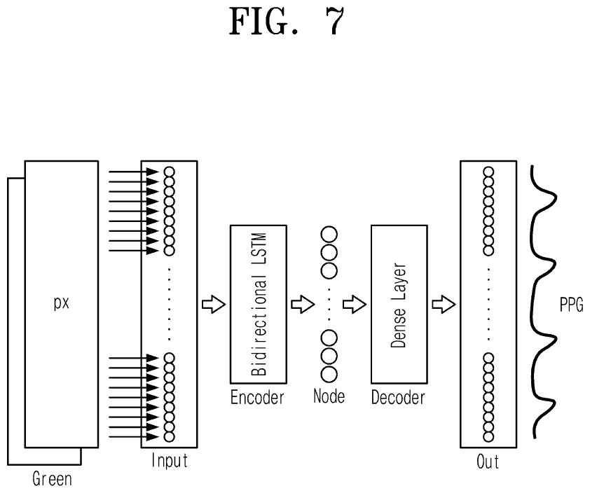

FIG. 7 illustrates a learning process of a model for image-based heart rate estimation according to the present disclosure.

As shown in FIG. 7, the data px obtained by preprocessing the image data and green image data may be used as inputs and the PPG signal may be set as a label (the correct answer), so that the inference model may be trained to reconstruct the signal to be identical to the PPG signal. The px and the green image data are used as the inputs, but other inputs may be used.

In one example, the augmented signal or the augmented data described with reference to FIG. 5 may be used as the label (the correct answer).

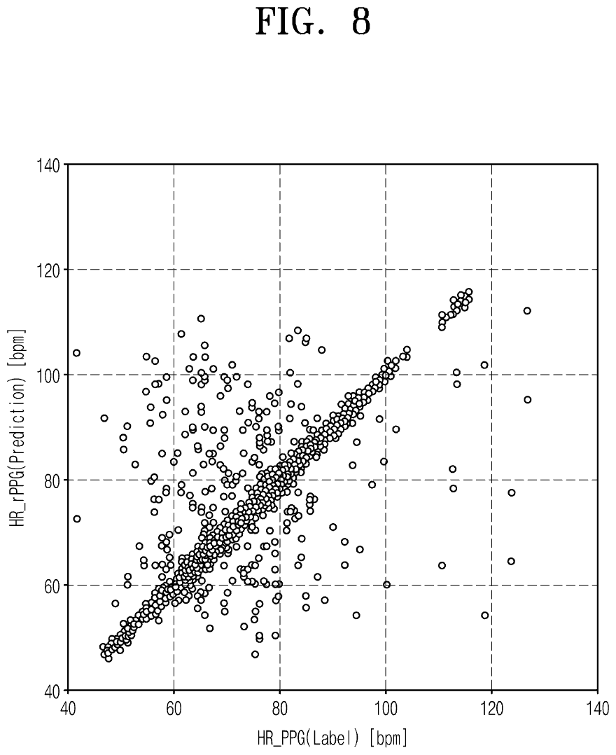

FIG. 8 shows a result of heart rate estimation according to the present disclosure.

FIG. 8 illustrates a result of the rPPG-based heart rate estimation of the object obtained from the plurality of images, as the result of training the inference model using the augmented signal or the augmented data described in FIG. 5. In FIG. 8, an x-axis represents an input of the PPG signal (the data set) corresponding to the known heart rate, and a y-axis represents the inference result obtained by processing the input in the inference model, that is, the estimated value of the heart rate.

Because the x-axis and the y-axis in FIG. 8 have the same dimension and scale, points distributed close to a straight line with a slope of 1 indicate accurate estimation results, and points distributed far from the straight line indicate errors.

Unlike FIG. 4, it may be seen that the error data is significantly reduced in the high heart rate section (equal to or higher than about 110 bpm).

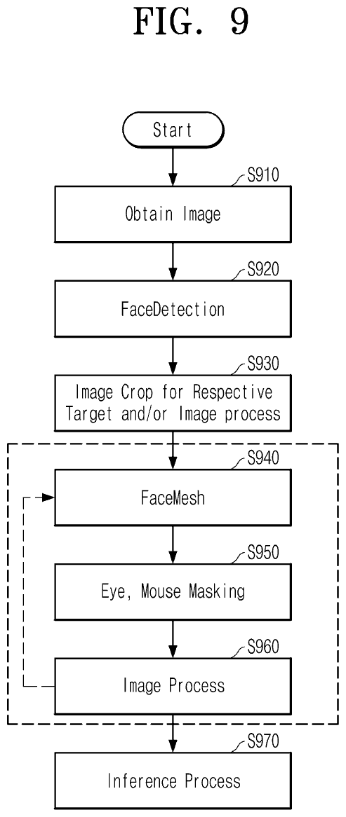

FIG. 9 is a flowchart of image-based heart rate estimation for multiple objects according to the present disclosure. The image-based heart rate estimation according to the present disclosure may be performed by the heart rate estimation apparatus or may be performed by the processor of the heart rate estimation apparatus. A description of the heart rate estimation apparatus will be made later with reference to FIG. 10. Hereinafter, it will be simply described that the apparatus 10 performs the heart rate estimation.

Unlike FIG. 6, FIG. 9 is for the heart rate estimation for the multiple objects, so that S930 is added, and S940 to S960 should be repeatedly performed. That is, S910 and S920 correspond to S610 and S620 in FIG. 6, and S970 also corresponds to S660 in FIG. 6. In addition, S940 to S960 also correspond to S630 to S650 in FIG. 6, but differ therefrom as they are performed for each object and thus are able to be repeated.

In conclusion, only the S930 needs to be described, and the remaining steps will be referred to the description of FIG. 6.

The apparatus 10 may crop a region of each object in the image and/or perform the image processing on the region of each object (S930).

The crop of the image region may be performed by distinguishing the face region of each object.

The image processing of the region of each object is to remove noise or improve image quality because the number of image pixels for each object is small when there are the plurality of objects, unlike when there is only a single object in the image. The image processing for each object region may include image processing such as scale up or pixel interpolation.

As the pixel interpolation technique, nearest neighbor interpolation, bilinear interpolation, bicubic interpolation, spline interpolation, Lanczos interpolation, Gaussian interpolation, and the like may be used, but the present disclosure may not be limited thereto.

In one example, in S930, the image processing may be optionally performed. That is, when a resolution or a noise state of the region of each object may have a significant effect on the result of the heart rate estimation, the image processing of the S930 may be performed. To this end, the apparatus 10 may determine whether the image processing for the region of each object is required.

The apparatus 10 may obtain an inference result using the image of the (face) region of each object that is image-processed as an input of the inference model (S970).

The heart rate estimation for the multiple objects may be utilized in a multi-user facility such as a hospital or an elderly protection facility. For example, in a multi-bed room, currently, each patient is required to wear a contact-type measuring apparatus for the heart rate, oxygen saturation, and the like to monitor the patient. However, based on the apparatus or the method for estimating the heart rates or the like for the multiple objects according to the present disclosure, the heart rates or the like of the multiple users may be estimated using the image sensor such as the camera, and may be tracked and monitored. Identification of the plurality of users needs to be accompanied.

FIG. 10 illustrates a block diagram of an apparatus for image-based cardiovascular-related biometric information estimation according to the present disclosure.

The apparatus 10 for the cardiovascular-related biometric information estimation may include a memory 100 and a processor 101.

The memory 100 may store data for estimating the cardiovascular biometric information. The data for estimating the cardiovascular biometric information may include the inference model described above.

In addition, the data for estimating the cardiovascular biometric information may include data obtained by learning the RGB data of the image and the PPG data corresponding to the RGB data. The RGB data of the image may include data obtained by preprocessing the RGB data of the image. In addition, the PPG data may correspond to a specific value of the cardiovascular biometric information.

In addition, the data for estimating the cardiovascular biometric information may include data obtained by learning virtual RGB data for a preset value or range of the cardiovascular biometric information and the virtual rPPG data corresponding to the virtual RGB data.

For example, when the cardiovascular biometric information is the heart rate, the preset range of the cardiovascular biometric information may be heart rate per minute equal to or lower than 50 or equal to or higher than 80.

The processor 101 may estimate the cardiovascular biometric information of the object in the obtained image using the data for estimating the cardiovascular biometric information.

In addition, the processor 101 may estimate the cardiovascular biometric information of the plurality of objects in the obtained image.

The processor 101 may output the cardiovascular biometric information of the object via a human-machine interface.

The processor 101 may crop the image region corresponding to each of the plurality of objects and/or scale up the cropped image region.

The processor 101 may identify the plurality of objects, and output the cardiovascular biometric information of the identified object via the human-machine interface.

The processor 101 may search for information matching the plurality of objects from user information stored in the memory. The user information stored in the memory may include feature information of the user.

As such, the processor 101 may identify the plurality of objects (or users). With the identification of the plurality of objects, the cardiovascular biometric information of each object may be tracked and monitored. In addition, when cardiovascular biometric information of a specific object exceeds or deviates from a preset threshold value or threshold range as well as when the tracking and the monitoring of each object are performed, a notification therefor may be output via the human-machine interface or a transceiver for wired/wireless communication.

In addition, the apparatus 10 may additionally include a human-machine interface (HMI) 102.

The human-machine interface (HMI) 102 may include means for providing a visual notification or an audible notification to the user or an administrator, and may include, for example, a display, a speaker (a buzzer), or an LED light. The notifications that the HMI 102 may provide may include the visual notification or the audible notification, as well as a haptic notification such as vibration, but the present disclosure may not be limited thereto.

Although not described with reference to FIG. 10, the apparatus 10 for estimating the cardiovascular-related information according to the present disclosure may perform the operations according to the present disclosure described above with reference to FIGS. 2, 5 to 7, and 9.

In another aspect of the present disclosure, the above-described proposals or inventive operations may also be provided as code capable of being implemented, performed, or executed by a “computer” (i.e., a comprehensive concept including a system-on-chip (SoC) or a processor (or microprocessor), a computer-readable storage medium including the aforementioned code, or a computer program product. The scope of the present disclosure may be extended to the code, the computer-readable storage medium including the code, or the computer program product.

The exemplary embodiments of the present disclosure have been provided to enable those skilled in the art related to the present disclosure to implement and practice the present disclosure. Although the above description has been provided with reference to the exemplary embodiments of the present disclosure, it will be understood by those skilled in the art that the present disclosure as set forth in the claims below may be modified and varied in various ways. Therefore, the present disclosure is intended to provide the broadest scope consistent with the principles and novel features disclosed herein, rather than being limited to the embodiments disclosed herein.

1. An apparatus for performing image-based cardiovascular biometric information estimation, the apparatus comprising:
a memory configured to store data for estimating the cardiovascular biometric information; and
a processor configured to estimate cardiovascular biometric information of an object in an obtained image using the data for estimating the cardiovascular biometric information.

2. The apparatus of claim 1, wherein the processor is configured to estimate cardiovascular biometric information of a plurality of objects in the obtained image.

3. The apparatus of claim 1, wherein the processor is configured to output the estimated cardiovascular biometric information of the object via a human-machine interface.

4. The apparatus of claim 2, wherein the processor is configured to:
crop an image region corresponding to each of the plurality of objects; and/or
scale up the cropped image region.

5. The apparatus of claim 2, wherein the processor is configured to identify the plurality of objects and output the estimated cardiovascular biometric information of the identified objects via a human-machine interface.

6. The apparatus of claim 5, wherein the processor is configured to search for information matching the plurality of objects from user information stored in the memory.

7. The apparatus of claim 5, wherein the processor is configured to transmit or output a notification message in response to that cardiovascular biometric information of one of the plurality of objects exceeds a preset threshold value or deviates from a threshold range.

8. The apparatus of claim 1, wherein the data for estimating the cardiovascular biometric information includes data obtained by learning RGB data of an image and photoplethysmography (PPG) data corresponding to the RGB data,
wherein the RGB data of the image includes data obtained by preprocessing the RGB data of the image,
wherein the PPG data corresponds to a particular value of the cardiovascular biometric information.

9. The apparatus of claim 1, wherein the data for estimating the cardiovascular biometric information estimation includes data obtained by learning virtual RGB data for a preset value or a preset range of cardiovascular biometric information and virtual remote photoplethysmography (rPPG) data corresponding to the virtual RGB data.

10. A method for performing image-based cardiovascular biometric information estimation, the method comprising:
obtaining an image;
detecting an object from the obtained image and obtaining a signal for a face region of the detected object; and
estimating cardiovascular biometric information of the detected object based on the obtained signal.

11. The method of claim 10, further comprising estimating cardiovascular biometric information of a plurality of objects in the obtained image.

12. The method of claim 10, further comprising outputting the estimated cardiovascular biometric information of the object via a human-machine interface.

13. The method of claim 11, further comprising:
cropping an image region corresponding to each of the plurality of objects; and/or
scaling up the cropped image region.

14. The method of claim 11, further comprising identifying the plurality of objects and outputting the estimated cardiovascular biometric information of the identified objects via a human-machine interface.

15. The method of claim 14, further comprising searching for information matching the plurality of objects from user information stored in a memory.

16. The method of claim 14, further comprising transmitting or outputting a notification message in response to that cardiovascular biometric information of one of the plurality of objects exceeds a preset threshold value or deviates from a threshold range.

17. The method of claim 10, wherein the data for estimating the cardiovascular biometric information estimation includes data obtained by learning RGB data of the image and PPG data corresponding to the RGB data,
wherein the RGB data of the image includes data obtained by preprocessing the RGB data of the image,
wherein the PPG data corresponds to a particular value of the cardiovascular biometric information.

18. The method of claim 10, wherein the data for estimating the cardiovascular biometric information estimation includes data obtained by learning virtual RGB data for a preset value or a preset range of cardiovascular biometric information and virtual remote photoplethysmography (rPPG) data corresponding to the virtual RGB data.

19. A computer-readable medium storing code to execute, by a computer or a processor, the method of claim 10.
